(19) United States
(12) Patent Application Publication
(10) Pub. No.: US 2018/0082563 A1
(43) Pub. Date: Mar. 22, 2018

(54) CAR SEAT MONITOR

(71) Applicants: Breianna Cristella, Philadelphia, PA (US); Scott Cristella, Philadelphia, PA (US)

(72) Inventors: Breianna Cristella, Philadelphia, PA (US); Scott Cristella, Philadelphia, PA (US)

(21) Appl. No.: 15/712,111

(22) Filed: Sep. 21, 2017

Related U.S. Application Data
(60) Provisional application No. 62/397,640, filed on Sep. 21, 2016.

(51) Int. Cl. G08B 21/04; A44B 11/25; A61B 5/11

(52) U.S. Cl. CPC G08B 21/0461; G08B 21/0415; B60R 22/105; A61B 5/1118; A44B 11/2569

(57) ABSTRACT

A monitoring system generates an alarm signal to alert a responsible adult or stimulates an infant when a signal from a sensor is indicative of a heightened risk of the infant dying such that intervention to save the infant is justified. The monitoring system is portable and is adapted for attachment to devices for carrying or seating infants for transport.

### CROSS-REFERENCE TO RELATED APPLICATIONS

This application claims the benefit of U.S. Provisional Application No. 62/397,640, filed on Sep. 21, 2016, which is incorporated herein by reference in its entirety.

### BACKGROUND OF THE INVENTION

Sudden infant death syndrome (SIDS) is the leading cause of death among infants between 1 month and 1 year of age according to the United States National Institutes of Health (NIH). More than 2,000 infants died of SIDS in 2010, the last year for which such statistics are available. SIDS deaths most often occur in infants between 1 month and 4 months of age, and about 90% of SIDS deaths occur before a baby reaches 6 months of age. However, SIDS deaths can occur anytime during an infant's first year of life. SIDS is a sudden and silent medical disorder that can happen to an infant who seems healthy. SIDS deaths are associated with the infants' sleeping time frame. (Source https://www.nichd.nih.gov/sts/about/SIDS/Pages/fastfacts.aspx.)

Infant sitting and carrying devices, such as infant car seats, infant carriers, and strollers, have an attendant risk of sleep associated infant death that has caused some medical professionals to discourage parent from using such devices for their babies long duration sleeping. (See http://www.medicaldaily.com/car-seats-are-no-place-naptime-study-finds-cribs-are-still-ideal-sleeping-infants-330682.) Although some monitoring systems intended to monitor babies in the home in the hope of reducing the risk of death from SIDS have been proposed, these systems are not particularly suitable for use with mobile infant sitting and carrying devices. The need persists for a monitoring system for reducing the risk of death from SIDS that is particularly suitable for use with mobile infant sitting and carrying devices.

### SUMMARY OF THE INVENTION

The present invention is directed to a monitoring system for reducing the risk of death from SIDS that is particularly suitable for use with mobile infant sitting and carrying devices. The monitoring system of the present invention can be integrated into the buckle systems used with the belt or strap systems for securing infants in devices for sitting, seating, or carrying infants. Examples of such devices include, without limitation, infant car seats, infant or baby carriers, combination car seat and carriers, strollers, buggies, and vehicles including motor vehicles.

Accordingly, it is an aspect of the present invention to provide a monitoring system for monitoring a physiological activity of an infant to help reduce the risk of death from SIDS of the infant, the monitoring system comprising:

a housing adapted for mounting to a device selected from an infant seating device, an infant carrying device, a vehicle, a belt or strap system for securing the infant in such devices, and combinations thereof;

a sensor supported by the housing for detecting the physiological activity and generating a signal corresponding to the physiological activity;

a processor for monitoring the signal from the sensor, the processor being programmed with at least one criterion for determining when the signal from the sensor is indicative of a heightened risk of the infant dying such that intervention to save the infant is justified;

output means communicating with the processor, the output means being selected from an alarm means and a stimulation means, the alarm generating a visual or audible alarm when the alarm means receives a signal from the processor indicative of the heightened risk of the infant dying so that a responsible adult can be alerted to the need for intervention to save the infant, and the stimulation means operating to impart stimulation to the infant that increases the likelihood that the infant will resume the physiological activity in a state that is considered within a normal range for the physiological activity when the stimulation means receives a signal from the processor indicative of the heightened risk of the infant dying; and

a battery for powering the monitoring system.

It is another aspect of the present invention to provide a monitoring system according to any of the other aspects of the present invention disclosed herein, wherein the housing is part of a buckle system of the belt or strap system.

It is still another aspect of the present invention to provide a monitoring system according to any of the other aspects of the present invention disclosed herein, wherein the housing includes at least one strap slot through which a portion of a belt or strap of the belt or strap system can extend.

It is yet another aspect of the present invention to provide a monitoring system according to any of the other aspects of the present invention disclosed herein, wherein the buckle system is a two part buckle system including a first part and a second part and wherein the housing is cooperatively formed by the first part and the second part of the buckle system.

It is yet another aspect of the present invention to provide a monitoring system according to any of the other aspects of the present invention disclosed herein, wherein the first part comprises a plug having two resilient prongs, each prong having a lateral protuberance, and the second part comprises a socket having one or more channels for receiving the prongs, wherein the socket has lateral openings, each of the lateral openings receiving the lateral protuberance of a respective one of the prongs when the first part and the second part of the buckle system are locked together.

It is yet another aspect of the present invention to provide a monitoring system according to any of the other aspects of the present invention disclosed herein, wherein the at least one criterion is for the signal from the sensor to indicate a lack of the physiological activity within the normal range for a predetermined period of time.

It is yet another aspect of the present invention to provide a monitoring system according to any of the other aspects of the present invention disclosed herein, wherein the predetermined period of time is from about 3 seconds to about 30 seconds.

It is yet another aspect of the present invention to provide a monitoring system according to any of the other aspects of the present invention disclosed herein, wherein the predetermined period of time is from about 3 seconds to about 5 seconds.

It is yet another aspect of the present invention to provide a monitoring system according to any of the other aspects of the present invention disclosed herein, wherein the output means is an alarm means selected from the group consisting of a red LED, an audible speaker, and a wireless LCD display unit for emitting an audible alarm or a visual alarm or both.

It is yet another aspect of the present invention to provide a monitoring system according to any of the other aspects of the present invention disclosed herein, wherein the output means is a stimulation means selected from a vibration motor and an audible speaker for emitting sounds that would be expected to awaken the infant.

It is yet another aspect of the present invention to provide a monitoring system according to any of the other aspects of the present invention disclosed herein, wherein the physiological activity being monitored is breathing and wherein the sensor comprises a motion detection device for sensing at least some movements of the infant's chest or abdomen resulting from inhalation and exhalation by the infant during breathing.

It is yet another aspect of the present invention to provide a monitoring system according to any of the other aspects of the present invention disclosed herein, wherein the at least one criterion is for the signal from the sensor to indicate a lack of normal breathing activity for a predetermined period of time.

It is yet another aspect of the present invention to provide a monitoring system according to any of the other aspects of the present invention disclosed herein, wherein the motion detection device comprises:

a sliding body supported by the housing for rectilinear movement between a retracted position and a fully projecting position relative to the housing such that the sliding body can assume a position relative to the housing anywhere between the retracted position and the fully projecting position, the sliding body being biased toward the fully projecting position, the sliding body engaging the chest or abdomen of the infant such that the position of the sliding body relative to the housing undergoes changes responsive to the at least some movements of the chest or abdomen of the infant during breathing; and

a variable resistor having a resistance value and being operably linked to the sliding body such that the resistance value of the variable resistor undergoes changes responsive to the at least some movements of the chest or abdomen of the infant during breathing,

wherein the changes in the resistance value is used in providing the signal generated by the sensor.

It is yet another aspect of the present invention to provide a monitoring system according to any of the other aspects of the present invention disclosed herein, wherein the motion detection device further comprises:

a rack positionally fixed relative to the sliding body such that the sliding body and the rack move rectilinearly relative to the housing as a unit; and

a pinion engaging the rack such that the pinion moves rotationally in response to rectilinear movement of the sliding body relative to the housing, the pinion being operably linked to the variable resistor such that the resistance value of the variable resistor undergoes changes responsive to rotational movement of the pinion.

It is yet another aspect of the present invention to provide a monitoring system according to any of the other aspects of the present invention disclosed herein, further comprising a multi-colored LED that is controlled to change color responsive to the amount of charge left in the battery.

It is yet another aspect of the present invention to provide a monitoring system according to any of the other aspects of the present invention disclosed herein, further comprising a green LED that is illuminated to indicate that the physiological activity is within the normal range.

It is yet another aspect of the present invention to provide a monitoring system according to any of the other aspects of the present invention disclosed herein, wherein the monitoring system automatically powers on when the first part and the second part of the buckle system are locked together.

It is yet another aspect of the present invention to provide a method for using a monitoring system according to any of the other aspects of the present invention disclosed herein, wherein the method comprises the steps of:

securing the infant in the device selected from an infant seating device, an infant carrying device, a vehicle, and combinations thereof;

monitoring the signal generated by the sensor; and

generating the signal to the output means to alert a responsible adult or to stimulate the infant when the signal from the sensor is indicative of a heightened risk of the infant dying such that intervention to save the infant is justified.

These and other aspects and advantages of the present invention will be further elucidated by the following Detailed Description, drawing figures, and Claims.

### BRIEF DESCRIPTION OF THE DRAWING FIGURES

FIG. 1 shows a front environmental view of the right hand part of a buckle incorporating an embodiment of the monitoring system of the present invention.

FIG. 2 shows a front environmental view of the left hand part of a buckle incorporating an embodiment of the monitoring system of the present invention.

FIG. 3 shows a top view of the assembled buckle incorporating an embodiment of the monitoring system of the present invention.

FIG. 4 shows a rear view of the assembled buckle incorporating an embodiment of the monitoring system of the present invention.

FIG. 5 is a diagrammatic view of the motion detection device of an embodiment of the monitoring system of the present invention.

FIG. 6 shows a graph of the waveform of the signal from the motion detection device of an embodiment of the monitoring system of the present invention corresponding to normal breathing.

FIG. 7 shows a flow chart of the method of operation of an embodiment of the monitoring system of the present invention.

FIG. 8 shows an isometric view of the LCD display of an embodiment of the monitoring system of the present invention.

FIG. 9 shows a top view of the LCD display of an embodiment of the monitoring system of the present invention.

FIG. 10 is a schematic component diagram of an embodiment of the monitoring system of the present invention.

### DETAILED DESCRIPTION

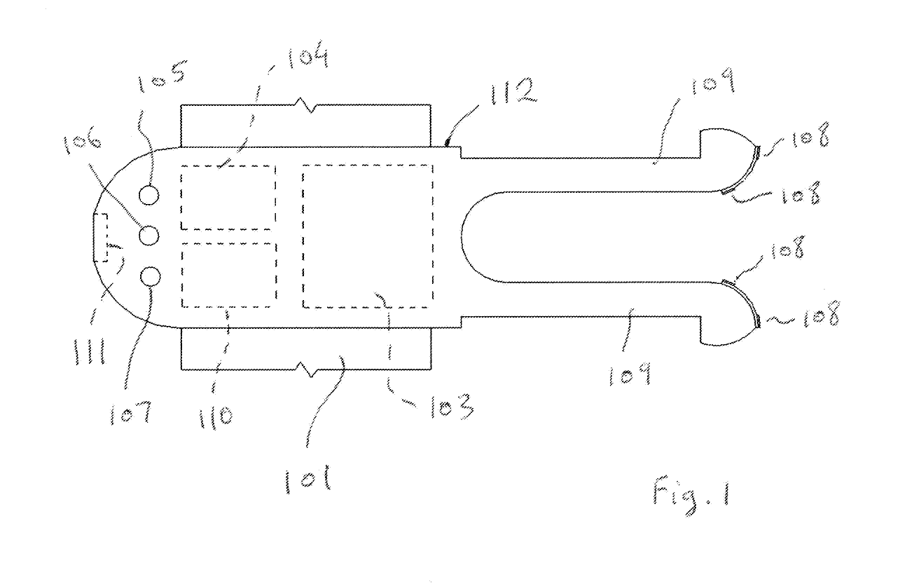

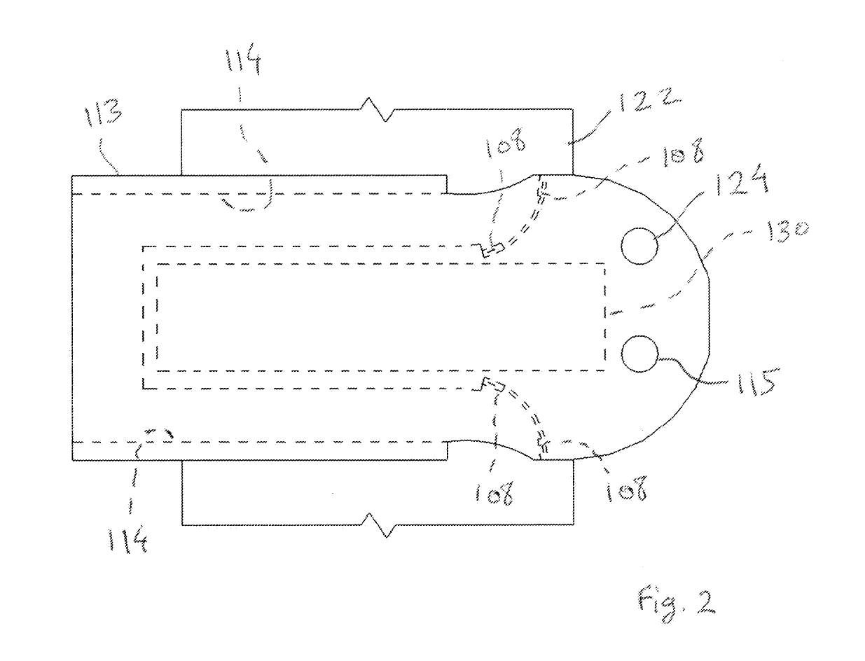

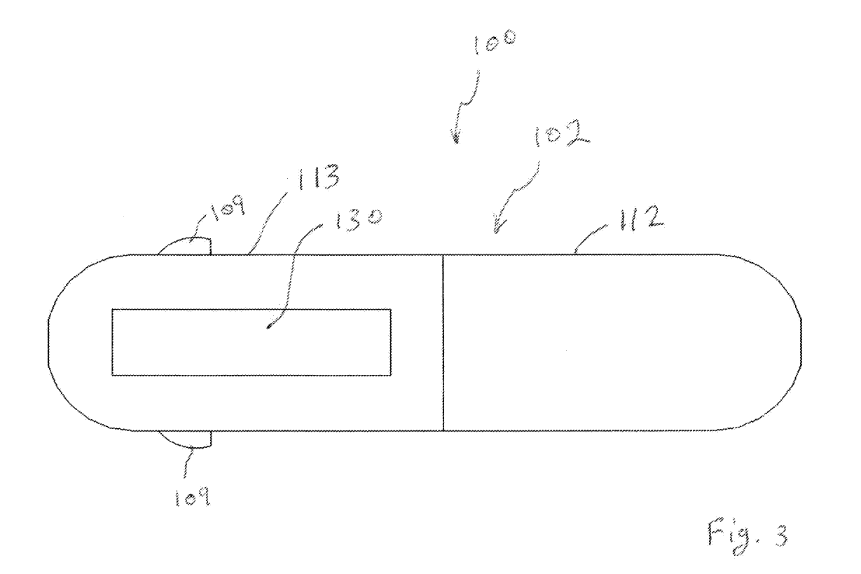

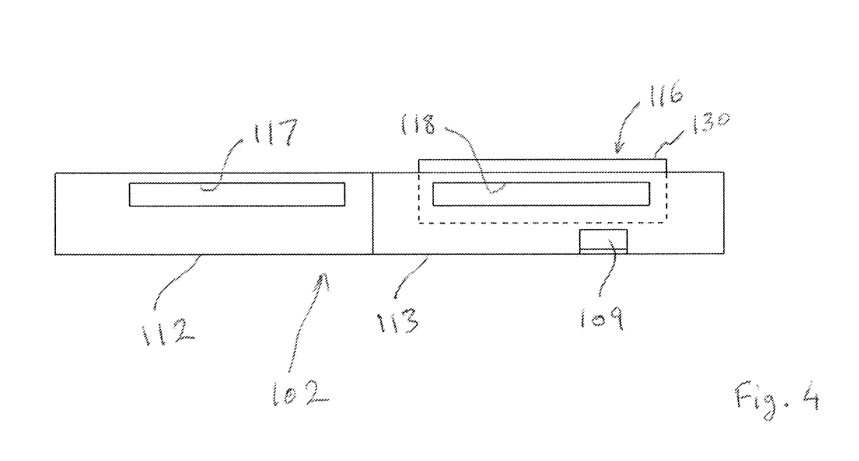

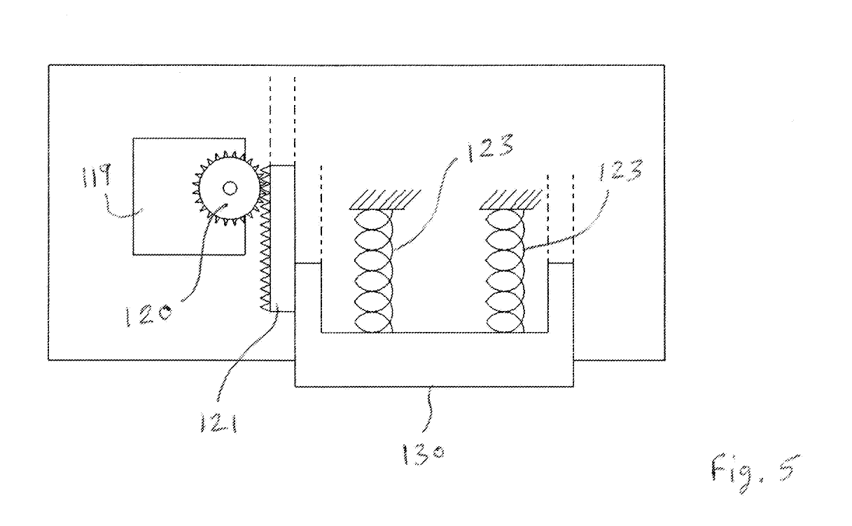

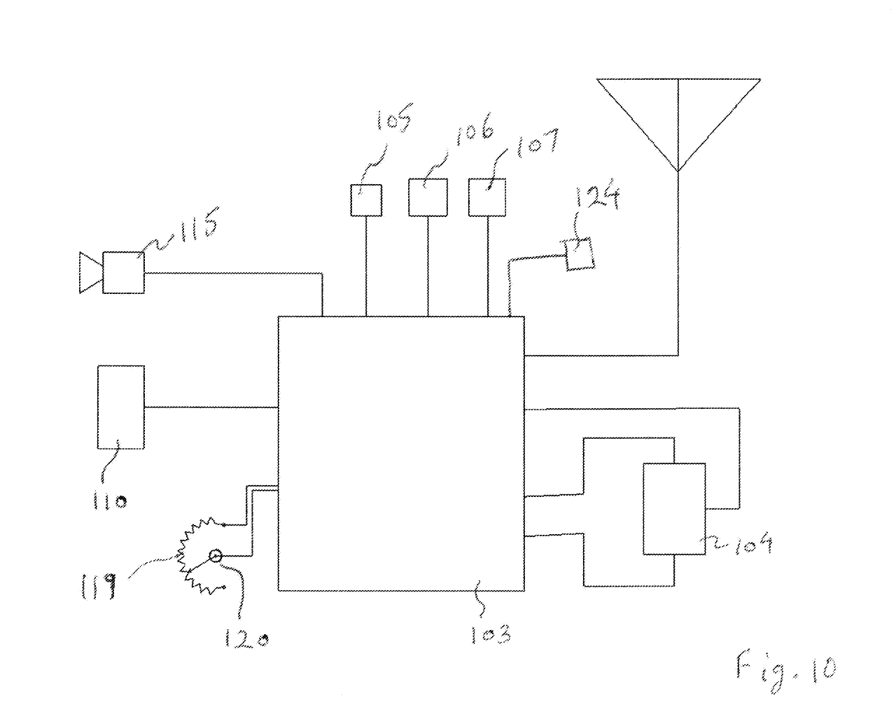

Referring to FIGS. 1-10, an illustrative embodiment a monitoring system 100 in accordance with the present invention can be seen. The monitoring system 100 is integrated into a car seat buckle 102. The car seat buckle 102 is more specifically an upper buckle for a chest strap. The car seat buckle 102 is a universal design car seat buckle made to monitor infant human chest movement. The car seat buckle 102 will have a movement monitor provided inside to help reduce the risk of death from Sudden Infant Death Syndrome (SIDS). Infant deaths from SIDS are believed to result from the stoppage of the infant's breathing during sleep. The monitoring system 100 is designed to monitor the movements of the infant's chest or abdomen corresponding to inhalation and exhalation by the infant during breathing. If the movements stop or are reduced to an abnormally small level for a predetermined period of time, the monitoring system will generate an alarm to alert a responsible adult to attend to the infant, or the monitoring system will generate sound, vibration, or electrical stimulation intended to stimulate the infant into resuming breathing.

In the situation that the adult human is unable to have direct visual contact with the infant, i.e. driving, in stroller, etc., and the infant human is in the car seat and buckled, the monitoring system 100 will notify the adult human of the infant human's current status.

The monitoring system 100 will have multiple status lights on the unit itself as well as transmitting status to a remote wireless display screen. These lights will be green for good or normal condition of the infant and red meaning to check the infant human.

The buckle 102 is also equipped with a vibration motor, audible speaker, and a rechargeable battery.

The drawing figures are not to scale and are diagrammatic sketches illustrating an exemplary embodiment of the monitoring system of the present invention. Broken lines show components inside the buckle that would otherwise be hidden from view in the views illustrated.

The buckle 102 is also referred to herein as a buckle system. The buckle 102 includes a right hand side buckle body 112 and left hand side buckle body 113. The right hand side buckle body 112 is also referred to herein as the right or first buckle part, while the left hand side buckle body 113 is also referred to herein as the left or second buckle part.

The right hand side buckle body 112 includes the locking tabs or prongs 109. The right hand side buckle body 112 houses components such as the processor/ECU 103, the battery 104, the LED indicator lights 105, 106, and 107, two of the electrical contact points 108, the vibration motor 110, and the battery charge port 111.

The left hand side buckle body 113 includes the internal channels 114 for the locking tabs or prongs 109. The left hand side buckle body 113 houses additional components such as the audible speaker 115, the motion detection device 116, and two of the electrical contact points 108. Note that the internal channels 114 are shown in broken lines.

The illustrative embodiment 100 of the monitoring system illustrates one type of motion detection device 116 developed for use with the monitoring system of the present invention. The motion detection device 116 uses a single rotation potentiometer 119 in conjunction with a rack and pinion style gear set 120, 121 and the sliding body 130 to generate a signal indicative of the movements of the human infant's chest/abdomen. The signal generated by the motion detection device 116 can then be used by the processor or ECU to monitor the movements of the human infant's chest/abdomen.

Housed within a standard looking upper chest buckle 102 on a typical infant car seat, infant carrier, or stroller, the monitoring system 100 will monitor movements of the human infant's chest/abdomen.

The potentiometer or variable resistor 119 is housed within to the main housing of the left buckle part 113. A potentiometer is by definition a “three terminal resistor with a sliding or rotating contact that forms an adjustable voltage divider. If only two terminals are used it acts as a variable resistor.” In the monitoring system 100, only two terminals of the potentiometer are used such that the potentiometer 119 functions as a variable resistor.

Attached to the shaft of the potentiometer 119 is a pinion gear 120 such that rotation of the pinion gear 120 will cause the sliding contact between one terminal of the potentiometer 119 to move relative to the resistive element of the potentiometer 119 and thus vary the resistance of the potentiometer 119. The pinion gear 120 is in engagement or in mesh with the rack gear 121. The rack gear 121 is fixed to the sliding body 130 such that the rack gear 121 and the sliding body 130 move rectilinearly as a unit as the sliding body 130 slides inward and outward relative to the left buckle part 113. The sliding body 130 is supported for rectilinear or sliding movement by the left buckle part 113 such that the sliding body 130 can slide into and out of the left buckle part 113, from the back side of the left buckle part, within a guide channel provided in the left buckle part.

When properly strapped into the infant car seat or like device, the inhalation and exhalation of the human infant will cause the sliding body 130 to move rectilinearly relative to the left buckle part 113.

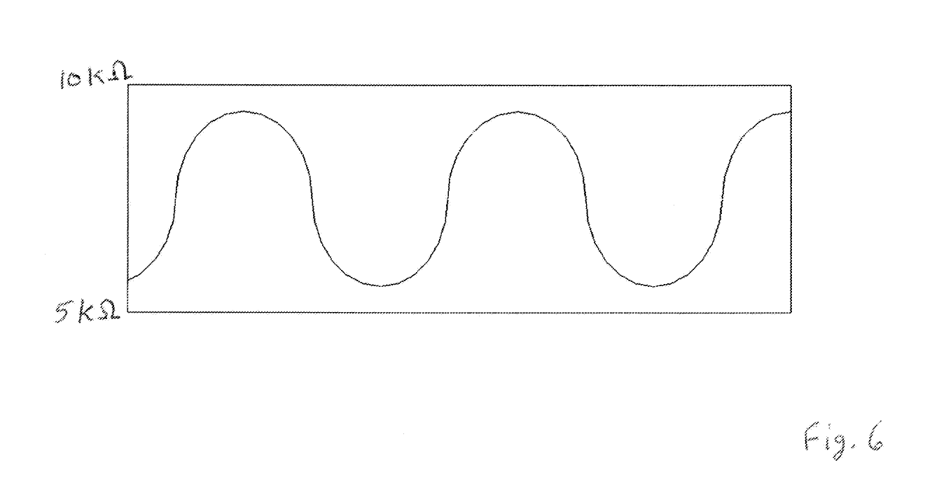

The sliding inward and outward of the sliding body 130 will cause the rack gear 121 to slide back and forth within its guide channels inside the left buckle part 113. The gear-like teeth on the rack 121 will interact with the teeth on pinion gear 120 causing the pinion gear 120 to rotate in place relative to the left buckle part 113 in response to the inward and outward rectilinear movement of the sliding body 130. Since the pinion gear 120 is attached to the potentiometer 119, rotation of the pinion gear 120 will cause the shaft of the potentiometer 119 to also rotate. This rotation produces a change in resistance of the potentiometer 119, which is the resistance between the terminal for the sliding contact of the potentiometer and the other terminal of the potentiometer that is being used for connection within the monitoring system 100. This resistance will increase and decrease as the human infant breathes. The increase and decrease in resistance will produce a signal having a waveform monitored by the processor 103. An example of the signal waveform corresponding to normal breathing activity is illustrated in FIG. 6.

The circuitry for generating the signal or waveform corresponding to the resistance of the potentiometer 119 is well known and will not be discussed here in detail. In the illustrated embodiment the circuitry for generating the signal or waveform corresponding to the resistance of the potentiometer 119 is internal to the processor 103 and is not shown. In such circuits, the two leads from the potentiometer 119 would be connected to a Wheatstone Bridge circuit such that the upper or lower part of one of the legs of the Wheatstone Bridge is formed by the potentiometer 119 as the resistance element. The voltage or current between the middle nodes of the two legs of the Wheatstone Bridge then generates the signal corresponding to the resistance of the potentiometer 119, which can then be converted to a digital signal using an analog-to-digital (A/D) converter in the processor 103 for further processing, monitoring, and/or storage by the processor 103.

The right hand side buckle body 112 has a cut out or slot 117 for the right hand side buckle strap 101, which is part of the belt or strap system of the infant car seat, infant carrier, stroller, or the like. This is a pre-formed slot in the right hand buckle part 112 to allow the right hand strap 101 to extend through the right buckle part for installing the buckle to the belt or strap system.

The left hand side buckle body 113 has a cut out or slot 118 for the left hand side buckle strap 122, which is part of the belt or strap system of the infant car seat, infant carrier, stroller, or the like. This is a pre-formed slot in the left hand buckle part 113 to allow the left hand strap 122 to extend through the left buckle part for installing the buckle to the belt or strap system.

The right hand side buckle strap 101 is a strong piece or strip of fabric that is part of the belt or strap system used to hold the human infant securely in the infant car seat, infant carrier, stroller, or the like. The left hand side buckle strap 122 is a strong piece or strip of fabric that is part of the belt or strap system used to hold the human infant securely in the infant car seat, infant carrier, stroller, or the like.

The processor or electronic control unit (ECU) 103 is the device used to receive and output data. The processor 103 receives waveform data generated using the variable resistor 119 to determine movements of the human infant in relation to breathing activity. The processor 103 outputs data in the form of control signals to illuminate the LED lights. The processor 103 outputs the on/off signal to the vibration motor 110 to provide vibratory stimulation to the infant only when stimulation is needed. The processor 103 outputs infant movements status to the wireless display screen 126. The processor 103 monitors the state of charge of the battery 104. The processor 103 outputs the on/off signal to the audible speaker 115. The processor 103 has a fifteen second delay timer once the buckle parts are buckled together before monitoring for movement.

The processor 103 includes a microcomputer that includes a CPU, memory for program, data, etc., input/output circuitry for communicating with and/or controlling all the peripheral devices that are also part of the processor 103. The peripheral devices within the processor 103 include all necessary circuits, including A/D convertor circuits, wireless communication circuits and antennae, power supply and power supply control circuits, etc., required for communicating with, controlling, receiving power from, and/or powering the peripheral devices outside the processor 103 such as the motion detection device including the sliding body 130 and the variable resistor 119 or other sensor or sensors, the battery 104, the wireless LCD display, the LED lights, the audible speaker 115, etc. The structures of the circuits and devices internal to the processor 103 are well known and are not discussed here in detail.

The battery 104 is preferably a rechargeable lithium ion battery. The battery 104 serves as an energy storage device for supplying power to all the monitoring system components housed in the right and left buckle parts 112, and 113, respectively. The battery charge port 111, provided in the right buckle body 112 for charging the battery 104, is preferably a micro-USB port. When the battery LED indicates that charging is required, a supply charger (not shown) is plugged into this port.

The green LED 105 Illuminates to indicate that movement corresponding to normal physiological activity such as breathing has been or is being detected. The red LED 106 Illuminates to indicate that movement, corresponding to normal physiological activity such as breathing, has not been detected over a specific period of time. The illumination of the red LED 106 indicates danger and that intervention to ensure that the infant is breathing is required. During the first five seconds after buckling the battery-shaped multi-colored LED 107 will flash; green indicating high battery life, yellow indicating low battery life, and red indicating that the battery needs to be charged. If desired, the processor 103 can be programmed to steadily illuminate the battery-shaped multi-colored LED 107 during operation of the monitoring system 100 with again green indicating high battery life, yellow indicating low battery life, and red indicating that the battery needs to be charged.

The electrical contact points 108 are a set of eight electrical contact points provide in the monitoring system 100. Four contact points 108 are contained in the left hand buckle 113, and four contact points 108 are contained in the right hand buckle 112. The two sets of four contact points 108 touch and make electrical contact when both buckle halves 112 and 113 lock together. When buckled, the four upper contact points 108 complete the battery circuit enabling the portion of the monitoring system 100 housed in the buckle 102 to power on. When buckled, the four lower contact points 108 complete the connection or circuit between the variable resistor 119 and the processor 103.

The locking tabs or resilient prongs 109 provide for the secure locking of the left and right buckle parts 113 and 112 together. Each prong 109 has a lateral protuberance. The left buckle part 113 includes a socket having one or more channels 114 for receiving the prongs 109 when the left and right buckle parts 113 and 112 are locked together. The socket or left buckle part 113 has lateral openings the protuberances at the ends of the prongs 109 when the left and right buckle parts 113 and 112 are locked together. When the left and right buckle parts 113 and 112 are locked together, each of the lateral openings of the left buckle part 113 receives the lateral protuberance of a respective one of the prongs 109. The internal channels 114 of the left buckle part 113 allow the locking prongs 109 to slide through the left buckle part 113 until the protuberances of the prongs 109 engage the lateral openings of the left buckle part 113. To release the right buckle part 112 from the left buckle part 113, the two prongs 109 are pressed toward each other by pressing the protuberances of the prongs 109, which are exposed through the lateral openings of the left buckle part 113, toward each other until the protuberances of the prongs 109 are disengaged from the lateral openings of the left buckle part 113.

The vibration motor 110 is a small electrical motor with an unbalanced weight attached to its shaft. As the shaft of the vibration motor 110 spins, the unbalanced weight spins also causing the buckle 102 to vibrate. The vibration motor 110 is tuned on under control of the processor 103 when the processor 103 determines that normal physiological activity, such as breathing, has not been detected over the specific period of time. The vibration is intended to stimulate the human infant to start breathing again in cases where a responsible adult may be unavailable to attend to the infant. As an alternative, a linear motor rectilinearly moving a weight relative to the buckle 102 may be used.

The audible speaker 115 is a small speaker activated by the processor 103 to alert an adult human of a no motion detected status, i.e. that movement, corresponding to normal physiological activity such as breathing, has not been detected over a predetermined period of time. Optionally, the audible speaker 115 may also be used to stimulate the human infant with loud sounds in an effort to stimulate the human infant to start breathing again.

The motion detection device 116 is one form of sensor that may be used with the monitoring system 100. The motion detection device 116 includes the potentiometer 119, the pinion gear 120, the rack gear 121, the sliding body 130, and tension springs 123. The pinion gear 120 is fixed in position relative to the left buckle part 113, is supported for rotation by the left buckle part 113, and is provided with gear teeth. The rack gear 121 is a straight, relatively flat bar provided with gear teeth and intended to rectilinearly move with the sliding body 130. The sliding body 130 is the part of the motion detection device 116 that physically contacts the human infant and is moved by the breathing motion of the infant's chest or abdomen. The sliding body 130 is attached to the rack gear 121 such that the sliding body 130 and the rack gear 121 rectilinearly move as a unit when installed in the left buckle part 113.

The tension springs 123 are positioned to act between the sliding body 130 and the left buckle body 113 such that the springs bias the sliding body 130 in an outward direction relative to the left buckle body 113. Accordingly, the springs 123 move the sliding body 130 outward from the left buckle body 113 when the human infant exhales. Thus, the sliding body 130 moves in response to the human infant inhaling and exhaling.

The pair button 124 is used to synchronize identification codes between the monitoring system portion housed in the buckle 102 and the wireless LCD display so that both devices have the capability to pair or link directly to each other, i.e. the devices mutually recognize each other as an authorized sender and receiver of data. This feature is required in the case of multiple such devices being used within same vehicle or within close proximity to another vehicle using such devices. One pair button 124 is provided on the buckle 102 and one pair button 124 is provided on the wireless LCD display 126.

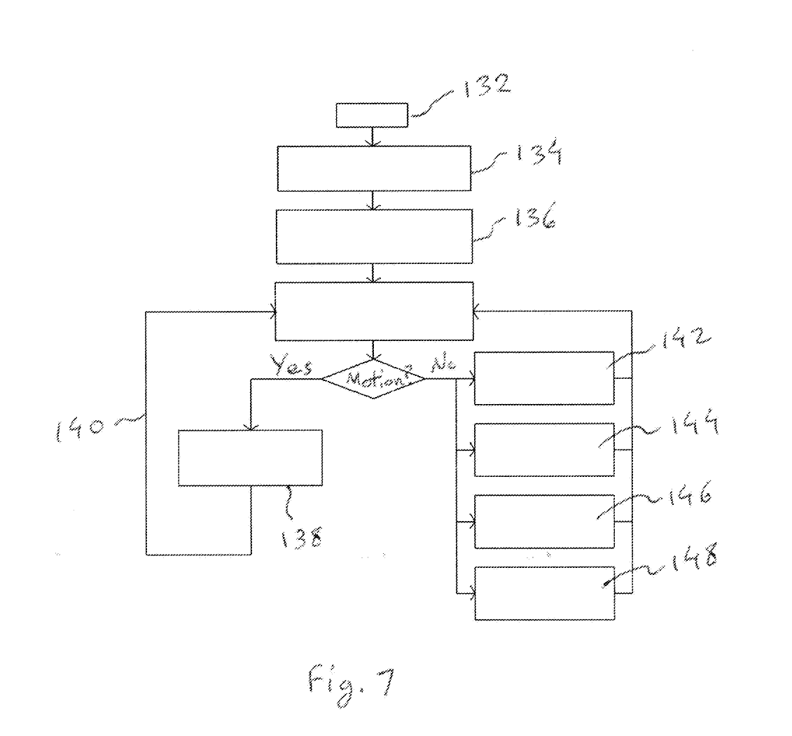

FIG. 7 shows a flow chart of the operation of the monitoring system of the present invention. Operation of the monitoring system begins when the unit is buckled (step 132), i.e. the right buckle part 112 and the left buckle part 113 are locked together. Simultaneously, the electrical circuits are completed between the processor 103 and the battery 104 and between the processor 103 and the variable resistor 119 through the electrical contact points 108.

The monitoring system 100, referred to as the unit in the flow chart, powers up, flashes the battery life indicator, starts the 15 second delay (step 134).

ECU 103 monitors motion detector 116 for changes in resistance of the variable resistor 119 and performs analysis (step 136).

If normal motion is detected, then the processor 103 flashes green LED on buckle and transmits signal to wireless monitor indicating a green or normal condition (step 138). Then the processor 103 continues monitoring for movement (step 140).

If no motion is detected, which means that the ECU 103 has analyzed the resistance pattern and determined no motion has been detected for the predetermined amount of time, then the processor/ECU 103 flashes the red LED on buckle 102 (step 142), transmits signal to wireless monitor 126 indicating a red or dangerous condition (step 144), and emits a loud audible siren using the audible speaker 115 (step 146). The ECU 103 also powers on vibration motor 110 (step 148).

The wireless monitor device 126, also referred to herein as the wireless LCD display, has a small framed LCD screen and its own processor (not shown). The wireless monitor device 126 has a rechargeable battery and a speaker. The wireless monitor device 126 has a clip 129 for grabbing on to an air duct vent louver in a vehicle provided on the back of the wireless monitor device housing 125. The wireless monitor device 126 is intended to receive information output by ECU 103 within the previously described motion detection buckle 102.

Upon receiving information of a motion detected signal from the ECU 103, the processor within the wireless monitor will output a command to the LCD display screen. The LCD screen will appear solid green, indicating a normal condition, until commanded otherwise. Upon receiving information that no motion has been detected for the predetermined period of time, as determined by the buckle ECU 103, the LCD display processor will command the LCD display screen to show red in color to indicate a dangerous condition for the infant requiring intervention. The LCD display processor will also command the audible speaker of the wireless LCD display 126 to emit a loud siren. The LCD display processor can also show visual messages:

1. Buckle battery low.

2. Wireless display battery low.

3. Out of range (when buckle 102 is out of range of the wireless LCD display 126).

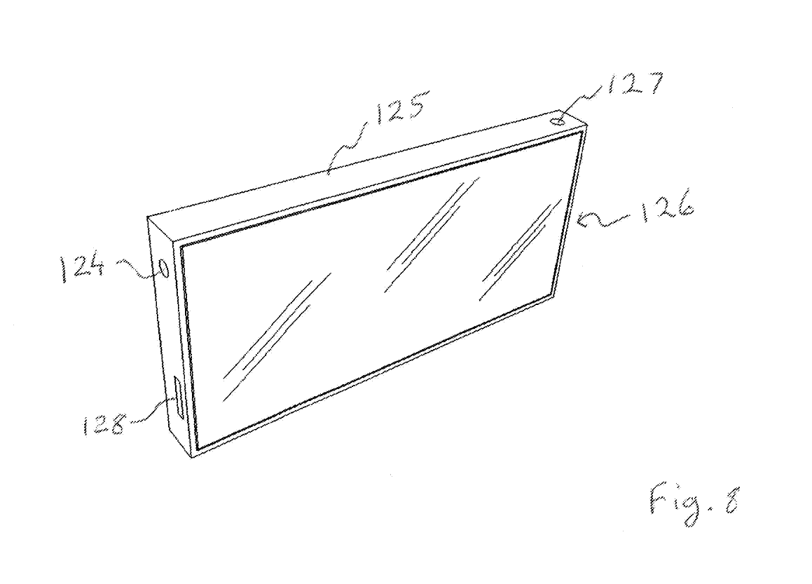

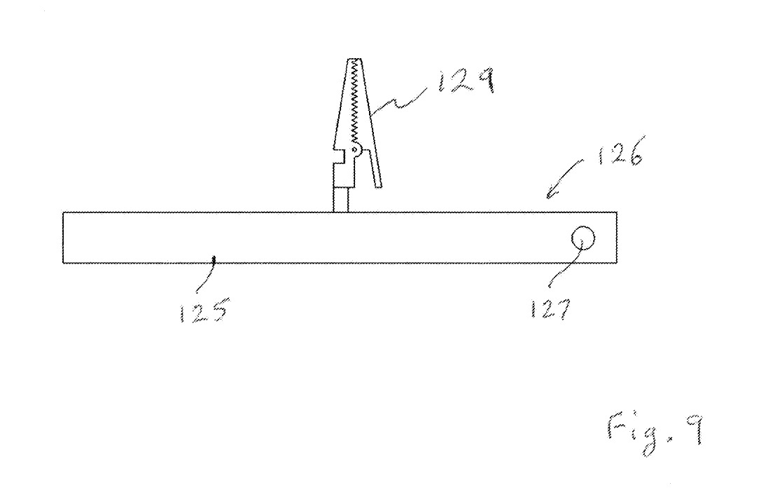

Referring to FIGS. 8 and 9, a diagrammatic depiction of an example of a wireless LCD display usable with the present invention can be seen. The sketches in FIGS. 8 and 9 are not to scale.

The body of LCD display 125 preferably has a hard plastic exterior that holds and contains all required components of the wireless LCD display 126. The Liquid crystal display of the wireless LCD display 126 can be used to display messages transmitted from buckle processor 103. The power button 127 is used to power the wireless LCD display 126 on and off. The charge port 128 is preferably a micro-USB port. When battery light of the wireless LCD display 126 indicates that charging is required, the supply charger (not shown) is plugged into this port. The air duct clip 129 is an alligator style clip affixed to the back of the LCD display body 125 for the purpose of attaching the wireless LCD display 126 to the air duct louver of an automobile or any other feasible support, i.e. stroller, shirt, belt loop, etc.

The wireless LCD display 126 can be a smart phone running an application program (app) specifically designed to implement all the required functionality of the wireless LCD display 126 in the smart phone.

As shown in FIG. 6, the output of the motion detection device is periodic during normal breathing. If breathing stops the output will become essentially flat or no periodicity is detectable for an abnormally long period of time. If such a condition persisted for a predetermined period of time then the alarm would be sounded by the monitoring system 100. Careful selection of the predetermined time period is called for in order to avoid any risk of damage to the infant, while reducing the occurrence of false alarms to an acceptable level.

At one minute of oxygen deprivation brain cell damage begins to occur in humans. Accordingly, if a child stops breathing, intervention should take place well before the one minute mark from the initial determination that the sensor signal does not indicate any breathing movement. It seems prudent that intervention should not be delayed for more than 30 seconds of a lack of breathing activity detection. In children from 6 months old up to and including 1 year of age, normal breathing can be as slow as 20 breaths per minute.

Therefore, a single normal breathing cycle may take three seconds in older infants. In children under six months, normal breathing can be as slow as 30 breaths per minute such that a single normal breathing cycle may take two seconds. Accordingly, a preferred range for the predetermined time period of a lack of breathing activity before an alarm is raised to intervene to save the infant would be from about 2 to about 30 seconds. An even more preferred range for the predetermined time period would be from about 3 to about 30 seconds, and a yet even more preferred range would be from about 3 to about 5 seconds.

Other motion sensors, i.e. motion detection devices, that may be used with the present invention, as alternatives to the motion detection device 116, include without limitation a hall-effect sensor in place of the potentiometer and rack and pinion arrangement, a laser doppler motion sensor in place of the potentiometer and rack and pinion arrangement, a laser doppler motion sensor directly measuring infant's chest/abdomen movement, a pressure sensor or transducer measuring pressure changes on the back of the buckle due to the infant's breathing, a strain gauge on the prongs 109 to measure changes in the strain in the prongs due to the infant's breathing, accelerometers or micro-miniature gyroscopes provided in the buckle 102. Also, other physiological activity or parameters can be monitored using appropriate sensors that would be indicative of possible danger to the infant and the need for intervention such as blood oxygen, heart rate, or brain activity (EEG). Appropriate sensors could be optical plethysmography sensors or electric field sensors.

Output means as used in the claims can be any means that can be used to warn adults or stimulate the infant and would include, for example, the LEDs 105, 106, the vibration motor 110, the audible speaker 115, and the wireless LCD display 126.

Alarm means as used in the claims can be any means that can be used to warn adults that the infant needs attendance, for example, the LEDs 105, 106, the audible speaker 115, and the wireless LCD display 126.

Stimulation means as used in the claims can be any means that can be used to stimulate the infant to start breathing again and would include, for example, the vibration motor 110, the audible speaker 115, and electrical stimulation means to provide, for example, mild shock.

It should be understood that the present invention is not limited to the specific embodiments described above, but includes any and all variations or modifications within the spirit and scope of the present invention as defined in the appended claims.

### LIST OF PARTS AND CORRESPONDING REFERENCE NUMERALS

Monitoring system 100

Right hand side buckle strap 101

Car seat buckle 102

Processor/Electronic Control Unit (ECU) 103

Battery 104

Green LED 105

Red LED 106

Battery-shaped, multi-color LED 107

Electrical contact points 108

Locking tabs 109

Vibration motor 110

Battery charge port 111

Right hand side buckle body 112

Left hand side buckle body 113

Internal channels for locking tabs 114

Audible Speaker 115

Motion detection device 116

Cut out slot for Right hand side buckle strap 117

Cut out slot for Left hand side buckle strap 118

Potentiometer/variable resistor 119

Pinion gear 120

Rack gear 121

Left hand side buckle strap 122

Tension springs 123

Pair button 124

Body of LCD display 125

LCD display 126

LCD display power button 127

LCD display charge port 128

Air duct clip 129

Sliding body 130

1. A monitoring system for monitoring a physiological activity of an infant to help reduce the risk of death from SIDS of the infant, the monitoring system comprising:
a housing adapted for mounting to a device selected from an infant seating device, an infant carrying device, a vehicle, a belt or strap system for securing the infant in such devices, and combinations thereof;
a sensor supported by said housing for detecting the physiological activity and generating a signal corresponding to the physiological activity;
a processor for monitoring said signal from said sensor, said processor being programmed with at least one criterion for determining when the signal from said sensor is indicative of a heightened risk of the infant dying such that intervention to save the infant is justified;
output means communicating with said processor, said output means being selected from an alarm means and a stimulation means, said alarm generating a visual or audible alarm when said alarm means receives a signal from said processor indicative of the heightened risk of the infant dying so that a responsible adult can be alerted to the need for intervention to save the infant, and said stimulation means operating to impart stimulation to the infant that increases the likelihood that the infant will resume the physiological activity in a state that is considered within a normal range for the physiological activity when said stimulation means receives a signal from said processor indicative of the heightened risk of the infant dying; and
a battery for powering the monitoring system.

2. The monitoring system of claim 1, wherein said housing is part of a buckle system of the belt or strap system.

3. The monitoring system of claim 2, wherein said housing includes at least one strap slot through which a portion of a belt or strap of the belt or strap system can extend.

4. The monitoring system of claim 2, wherein said buckle system is a two part buckle system including a first part and a second part and wherein said housing is cooperatively formed by said first part and said second part of said buckle system.

5. The monitoring system of claim 4, wherein said first part comprises a plug having two resilient prongs, each prong having a lateral protuberance, and said second part comprises a socket having one or more channels for receiving said prongs, wherein said socket has lateral openings, each of said lateral openings receiving said lateral protuberance of a respective one of said prongs when said first part and said second part of said buckle system are locked together.

6. The monitoring system of claim 1, wherein said at least one criterion is for the signal from said sensor to indicate a lack of the physiological activity within the normal range for a predetermined period of time.

7. The monitoring system of claim 6, wherein the predetermined period of time is from about 3 seconds to about 30 seconds.

8. The monitoring system of claim 6, wherein the predetermined period of time is from about 3 seconds to about 5 seconds.

9. The monitoring system of claim 1, wherein said output means is an alarm means selected from the group consisting of a red LED, an audible speaker, and a wireless LCD display unit for emitting an audible alarm or a visual alarm or both.

10. The monitoring system of claim 1, wherein said output means is a stimulation means selected from a vibration motor and an audible speaker for emitting sounds that would be expected to awaken the infant.

11. The monitoring system of claim 1, wherein the physiological activity being monitored is breathing and wherein said sensor comprises a motion detection device for sensing at least some movements of the infant's chest or abdomen resulting from inhalation and exhalation by the infant during breathing.

12. The monitoring system of claim 11, wherein said at least one criterion is for the signal from said sensor to indicate a lack of normal breathing activity for a predetermined period of time.

13. The monitoring system of claim 12, wherein the predetermined period of time is from about 3 seconds to about 30 seconds.

14. The monitoring system of claim 12, wherein the predetermined period of time is from about 3 seconds to about 5 seconds.

15. The monitoring system of claim 11, wherein said motion detection device comprises:
a sliding body supported by said housing for rectilinear movement between a retracted position and a fully projecting position relative to said housing such that said sliding body can assume a position relative to said housing anywhere between said retracted position and said fully projecting position, said sliding body being biased toward said fully projecting position, said sliding body engaging the chest or abdomen of the infant such that the position of said sliding body relative to said housing undergoes changes responsive to the at least some movements of the chest or abdomen of the infant during breathing; and
a variable resistor having a resistance value and being operably linked to said sliding body such that the resistance value of said variable resistor undergoes changes responsive to the at least some movements of the chest or abdomen of the infant during breathing,
wherein the changes in the resistance value is used in providing the signal generated by the sensor.

16. The monitoring system of claim 15, wherein said motion detection device further comprises:
a rack positionally fixed relative to said sliding body such that said sliding body and said rack move rectilinearly relative to said housing as a unit; and
a pinion engaging said rack such that said pinion moves rotationally in response to rectilinear movement of said sliding body relative to said housing, said pinion being operably linked to said variable resistor such that the resistance value of said variable resistor undergoes changes responsive to rotational movement of said pinion.

17. The monitoring system of claim 1, further comprising a multi-colored LED that is controlled to change color responsive to the amount of charge left in the battery.

18. The monitoring system of claim 1, further comprising a green LED that is illuminated to indicate that the physiological activity is within the normal range.

19. The monitoring system of claim 4, wherein the monitoring system automatically powers on when said first part and said second part of said buckle system are locked together.

20. A method for using the monitoring system of claim 1, the method comprising the steps of:
securing the infant in the device selected from an infant seating device, an infant carrying device, a vehicle, and combinations thereof;
monitoring the signal generated by the sensor; and
generating the signal to the output means to alert a responsible adult or to stimulate the infant when the signal from said sensor is indicative of a heightened risk of the infant dying such that intervention to save the infant is justified.
